(19) United States
(12) Patent Application Publication
(10) Pub. No.: US 2018/0112175 A1
(43) Pub. Date: Apr. 26, 2018

(54) METHOD FOR PRODUCING AN ORGANIC ACID USING EUGLENA

(71) Applicants: Riken, Wako-shi, Saitama (JP); eugiena Co,. Ltd., Minato-ku, Tokyo (JP)

(72) Inventors: Takashi Osanai, Wako-shi, Saitama (JP); Masami Hirai, Wako-shi, Saitama (JP); Hiroko IIjima, Wako-shi, Saitama (JP); Yuka Nakaya, Wako-shi, Saitama (JP); Kengo Suzuki, Minato-ku, Tokyo (JP); Osamu Iwata, Minato-ku, Tokyo (JP); Ayaka Nakashima, Minato-ku, Tokyo (JP)

(73) Assignees: Riken, Wako-shi, Saitama (JP); euglena Co., Ltd., Minato-ku, Tokyo (JP)

(21) Appl. No.: 15/571,280

(22) PCT Filed: May 6, 2016
(86) PCT No.: PCT/JP2016/063656

§ 371 Date: Nov. 2, 2017

(30) Foreign Application Priority Data
Aug. 8, 2015 (JP) 2015-095993

(51) Int. Cl. C12N 1/10; C12P 7/46

(52) U.S. Cl. CPC C12N 1/10; C12P 7/46

(57) ABSTRACT

The present invention provides a method for producing an organic acid using algal biomass, The present invention pertains to the production of organic acids such as succinic acid using Euglena , by means of a method containing; either a nitrogen deficient culture step in which Euglena is cultured. aerobically under nitrogen-deficient conditions or a heterotrophic culture step in which Euglena is cultured aerobically using a culture medium containing a carbon source; and an anaerobic culture step in which the culture product obtained in the nitrogen-deficient culture step is incubated under anaerobic conditions.

### TECHNICAL FIELD

The present invention relates to: a method for producing an organic acid including, as a representative example, succinic acid that is useful as a raw material for polybutylene succinate (PBS), using Euglena ; and a culture product of Euglena obtained by the aforementioned method.

### BACKGROUND ART

In recent years, the research and development of a biomass plastic comprising biomass as a raw material and a biodegradable plastic decomposed by the action of microorganisms in the nature have been advanced as novel plastic materials replaced for conventional petroleum-based plastics. As a biodegradable biomass plastic having the properties of both the biomass plastic and the biodegradable plastic, for example, polylactic acid (PLA), starch resin, polyhydroxy alkanoate (PHA), and polybutylene succinate (PBS) have been known.

PBS has been known as a highly useful biodegradable biomass plastic, which is synthesized by a condensation reaction of succinic acid with 1,4-butanediol. Succinic acid used as a raw material for PBS had been conventionally synthesized from petroleum as a raw material. Recently, such succinic acid has also been produced using carbohydrate derived from corn or sugarcane according to fermentation with heterotrophic bacteria. However, if such a food plant is used as a raw material, it may cause a competition with demand for foodstuff and animal feed, and it may be problematic in terms of stable supply. Accordingly, it is preferable to produce succinic acid from biomass used as a raw material, which does not compete with existing food production.

As such biomass, algae have recently attracted attention. Among others, it has been known that Euglena (Midorimushi) has an ability to produce various compounds as a result of photosynthesis, and that such Euglena can also be used in fuel production, For example, Patent Literature 1 discloses a method for producing Euglena comprising a large amount of wax ester used as a raw material for biofuel.

### CITATION LIST

### Patent Literature

Patent Literature 1: JP Patent Publication (Kokai) No, 2013.-153730 A

### SUMMARY OF INVENTION

### Technical Problem

Although various types of algae have been studied so far, the types of algae that produce a large amount of organic acid such as succinic acid, culture conditions thereof, and the like have not yet been discovered. It is an object of the present invention to provide a method for producing an organic acid such as succinic acid, using algae.

### Solution to Problem

The present inventors have studied various types of algae. As a result, the inventors have found that a culture product comprising a large amount of organic acids can be obtained by culturing Euglena under nitrogen-deficient conditions, or using a medium containing an organic or inorganic carbon source under heterotrophic conditions, and then incubating the culture product under anaerobic conditions. The features of the present invention are as follows.
(1) A method for producing an organic acid using Euglena , which comprises a nitrogen-deficient culture step of aerobically culturing Euglena under nitrogen-deficient conditions, and an anaerobic culture step of incubating a culture product obtained in the nitrogen-deficient culture step under anaerobic conditions.
(2) The method according to the above (1), wherein the nitrogen-deficient culture step is carried out for 3 days or more.
(3) A method for producing an organic acid using Euglena , which comprises a heterotrophic culture step of aerobically culturing Euglena using a medium containing a carbon source, and an anaerobic culture step of incubating a culture product obtained in the heterotrophic culture step under anaerobic conditions.
(4) The method according to any one of the above (1) to (3), which further comprises a step of collecting a culture medium containing the organic acid from the culture product obtained in the anaerobic culture step.
(5) The method according to any one of the above (1) to (4), which further comprises a step of concentrating a Euglena cell body contained in the culture product obtained in the nitrogen-deficient culture step or the heterotrophic culture step, before performing the anaerobic culture step.
(6) The method according to any one of the above (1) to (5), wherein the Euglena is wild-type Euglena.
(7) The method according to any one of the above (1) to (6), wherein the organic acid is succinic acid.
(8) A culture product obtained by culturing Euglena , comprising an organic acid produced by the Euglena in a concentration of 100 mg/L, or more.
(9) The culture product according to the above (8), wherein the organic acid is succinic acid.
(10) The culture product according to the above (8) or (9), which is a culture medium obtained after the Euglena has been cultured.
(11) The culture product according to any one of the above (8) to (10), which is obtained by culturing the Euglena according to a method comprising a nitrogen-deficient culture step of aerobically culturing Euglena under nitrogen-deficient conditions for 3 days or more, and an anaerobic culture step of incubating a culture product obtained in the nitrogen-deficient culture step under anaerobic conditions.
(12) The culture product according to any one of the above (8) to (10), which is obtained by culturing the Euglena according to a method comprising a heterotrophic culture step of aerobically culturing the Euglena using a medium containing a carbon source, and an anaerobic culture step of incubating a culture product obtained in the heterotrophic culture step under anaerobic conditions.
(13) The culture product according to any one of the above (8) to (1), wherein the Euglena is wild-type Euglena ,
(14) A Euglena cell body obtained by a method comprising a nitrogen-deficient culture step of aerobically culturing Euglena under nitrogen-deficient conditions for 3 days or more, an anaerobic culture step of incubating a culture product obtained in the nitrogen-deficient culture step under anaerobic conditions, and a step of collecting a Euglena cell body from the culture product obtained in the anaerobic culture step,
(15) A Euglena cell body obtained by a method comprising a heterotrophic culture step of aerobically culturing Euglena using a medium containing a carbon source, an anaerobic culture step of incubating a culture product obtained in the heterotrophic culture step under anaerobic conditions, and a step of collecting a Euglena cell body from the culture product obtained in the anaerobic culture step.

### Advantageous Effects of Invention

According to the present invention, it becomes possible to produce an organic acid including, as a representative example, succinic acid that is useful as a raw material for PBS and the like, using Euglena that is an algal biomass. In addition, simultaneously with production of an organic acid, a useful Euglena cell body can also be obtained.

The present description includes the contents as disclosed in the description, claims and drawings of Japanese Patent Application No. 2015-095993, which is a priority document of the present application.

### DESCRIPTION OF EMBODIMENTS

The method of the present invention is characterized in that it comprises either a nitrogen-deficient culture step of aerobically culturing Euglena under nitrogen-deficient conditions, or a heterotrophic culture step of aerobically culturing Euglena using a medium containing a carbon source, and an anaerobic culture step of incubating the culture product obtained in the aforementioned step under anaerobic conditions. The organic acid such as succinic acid, which is a compound of interest, is comprised in the culture product obtained in the anaerobic culture step. It is to be noted that, in the present description, the term “culture product” is a concept including all products obtained by culturing Euglena. More specifically, when Euglena is cultured in a culture medium placed in a culture tank, the “culture product” is a concept including all of culture media containing Euglena cell bodies and substances produced by the Euglena, namely, all of the contents in the culture tank.

Euglena (Japanese name: Midorimushi) is a generic name for microorganisms belonging to genus Euglena. It has been known that Euglena is algae having motility, and that a majority thereof has both animal properties including flagellar movement, and plant properties of having chloroplast to carry out photosynthesis. Specific examples of Euglena include Euglena acus, Euglena caudata, Euglena chadefaudii, Euglena deses, Euglena gracilis, Euglena granulata, Euglena intermedia, Euglena mutabilis, Euglena oxyuris, Euglena proxima, Euglena spirogyra, Euglena viridis, and Euglena vermiformis. Among these, Euglena gracilis, which has been conventionally widely utilized in studies, can be preferably used in the method of the present invention.

If wild-type Euglena, which has not been undergone genetic recombination, is used herein, there is no need for considering genetic hybridization in the nature, upon the culture thereof. Thus, Euglena can be cultured even outside, and costs for the culture can be preferably reduced. However, the type of the Euglena used herein is not limited thereto, and Euglena, which has been genetically modified to produce an organic acid such as succinic acid in a larger amount than usual, may also be used.

### (Nitrogen-Deficient Culture Step and Heterotrophic Culture Step)

In the nitrogen-deficient culture step, Euglena is aerobically cultured under nitrogen-deficient conditions, for example, using a nitrogen-deficient medium. When Euglena is cultured under nitrogen-deficient conditions, initially, a nitrogen source accumulated in the Euglena cell body is utilized. However, after such a nitrogen source has been all assimilated, the Euglena falls in a nitrogen-deficient state. It is considered that corresponding to such stress, the Euglena produces an organic acid such as succinic acid, and accumulates it in the cell body thereof. The nitrogen-deficient culture period is 3 days or more. If the nitrogen-deficient culture period is set at preferably 4 days or more, particularly preferably 5 days or more, or further, 6 days or more, it is considered that a large amount of an organic acid is preferably accumulated.

As conditions for the aerobic culture of Euglena using a nitrogen-deficient medium, conditions, which have been known regarding the conventional culture method of Euglena, can be used. The culture temperature is preferably set in the range of generally 20° C. to 34° C. for the efficient growth of Euglena. In addition, it is preferable to add air into the medium by bubbling. In particular, since Euglena assimilates CO₂, it is more preferable to add air containing 1% to 5% CO₂ into the medium by bubbling. The aerobic culture is preferably carried out under light irradiation (under a light condition), and is more preferably carried out under light-dark cycle conditions, and in particular, under light-dark cycle conditions in accordance with the circadian rhythm (e.g., a light-dark cycle of 12 hours). The light intensity under a light condition is preferably set in the range of 30 to 200 μmol m⁻²s⁻¹. The pH of the medium is preferably set in the range of pH 3 to 8.

For the culture of Euglena, a modified medium, which is obtained by partially changing the composition of a known medium such as, for example, Cramer-Myers medium (CM medium), Hutner medium, Koren-Hutner medium, or AY medium, can be used. Such modification of the medium includes the removal of a nitrogen source to create nitrogen-deficient conditions. In the case of the CM medium for example, nitrogen-deficient conditions can be created by removing ammonium phosphate (NH₄)₂HPO₄) from the composition thereof. It is to be noted that the term “nitrogen-deficient conditions” means a state in which the concentration of nitrogen in the medium is 0.1 mmol/L or less, and in particular, 0.01 mmol/L or less, relative to nitrogen atoms.

Modification of the medium used in the culture of Euglena may also include addition of a nutrient source for promoting the growth of Euglena, and addition of a carbon source that can be a raw material for the biosynthesis of an organic acid. The culture of Euglena may be carried out using a medium supplemented with a carbon source, under completely heterotrophic conditions, or under conditions in which photosynthesis can be used in combination with a carbon source added to the medium (in the present description, such conditions, in which photosynthesis is used in combination, are also included in the heterotrophic conditions). By culturing Euglena under heterotrophic conditions, a larger amount of an organic acid is accumulated, than in the case of culturing Euglena under autotrophic conditions. The aforementioned nitrogen-deficient culture step can be replaced with a heterotrophic culture step of culturing Euglena under heterotrophic conditions. The heterotrophic culture step may also be carried out under nitrogen-sufficient conditions, in which the nitrogen source is not removed from the medium. The heterotrophic culture step can be carried out in the same manner as the nitrogen-deficient culture step, with the exception that a medium having a different configuration is used.

Examples of the carbon source added to the medium include: sugars such as glucose or fructose; alcohols such as ethanol; organic acids or salts thereof, including carboxylic acids such as acetic acid or malic acid, or amino acids such as glutamic acid; and inorganic carbon compounds such as sodium hydrogen carbonate. For example, when ethanol is added in an amount of 0.5% to 1.0% by volume to the medium, it leads to promotion of the growth of Euglena. Moreover, as nutrient sources other than the carbon source, the salts of alkaline metal or alkaline-earth metal, such as potassium chloride or magnesium chloride, may be added to the medium. As a carbon source added to the medium, sugars and organic acids are preferable, and among them, glucose, and acetate and the salt thereof are particularly limited. From the viewpoint of keeping balance between the carbon source and other components contained in the medium, the carbon source is preferably added to the medium, in a concentration range of 1 to 200 mM, preferably 1 to 100 and particularly preferably 1 to 20 mM.

As Euglena used in the nitrogen-deficient culture step or the heterotrophic culture step, Euglena, which has been previously pre-cultured using a common medium that is not a nitrogen-deficient medium, is used. A culture medium, in which Euglena that has been pre-cultured preferably for 1 day, more preferably for 2 days or more, and particularly preferable for 3 days for more is dispersed, is collected, is then centrifuged, and as necessary, is washed with deionized water. Thereafter, the resulting precipitate is added to a medium or the like, and the obtained mixture is then left under nitrogen-deficient conditions or under heterotrophic conditions. From the viewpoint of the growth efficiency of Euglena, and further, the production efficiency of an organic acid, the concentration of the Euglena cell body at the initiation of the culture step is preferably set at 10 to 20 g/L, and the concentration of the Euglena cell body at the completion of the culture is preferably set at 20 to 50 g/L.

It is to be noted that, in the present description, the weight of a Euglena cell body means a dry weight, unless otherwise specified. The dry weight of a Euglena cell body is obtained by sampling a predetermined amount of culture medium in which Euglena is dispersed, then subjecting the culture medium to steps, such as centrifugation, washing and freeze-drying, so as to remove the water content, and then measuring the resulting Euglena cell body. Accordingly, the concentration of a Euglena cell body indicated at a unit of g/L means the dry weight of a Euglena cell body contained in 1 L of the culture medium.

### (Anaerobic Culture Step)

In the anaerobic culture step, a culture product obtained in the above-described nitrogen-deficient culture step or heterotrophic culture step is incubated under anaerobic conditions. Such incubation under anaerobic conditions is preferably carried out under a dark condition. The term “dark condition” is used herein to specifically mean a weak light condition in which the light intensity is 1 μmol m⁻²s⁻¹ or less, or a completely dark condition without light.

The “anaerobic culture” is a step, which is also called “anaerobic fermentation.” The anaerobic culture means a catabolism, in which, in order to acquire energy under anaerobic conditions, Euglena oxidizes organic matters by anaerobic respiration and then produces an organic acid such as succinic acid and carbon dioxide. Moreover, the “anaerobic conditions” are conditions, in which the oxygen concentration is lower than the minimum oxygen amount in which Euglena can breathe by aerobic respiration, and in which oxygen is substantially not present. The anaerobic conditions mean conditions, in which the oxygen concentration is generally 1% or less, preferably 0.5% or less, particularly preferably 0.2% or less, and further, completely oxygen-free conditions (wherein the oxygen amount is a detection limit or less). When Euglena is cultured under aerobic and nitrogen-deficient conditions, or under heterotrophic conditions, it accumulates an organic acid such as succinic acid in the cells thereof it is considered that, when such Euglena is cultured under anaerobic and dark conditions, the biosynthesis of an organic acid such as succinic acid is further promoted, and the Euglena then releases the organic acid to the outside of the cells. Anaerobic conditions can be achieved by adjusting the oxygen concentration in the gaseous phase in the culture tank. For instance, such anaerobic conditions can be achieved by substituting the gas in the gaseous phase with inert gas such as nitrogen or argon.

With regard to the culture product to be subjected to the anaerobic culture, if a supernatant is discarded after completion of the centrifugation and the precipitated Euglena cell body is dispersed, for example, in a small amount of fresh medium, so as to previously concentrate the Euglena cell body, the Euglena cell body can easily release an organic acid into the culture medium, and thus, the content of the organic acid in the culture medium after completion of the anaerobic culture step preferably increases. If the concentration is carried out such that the ratio of the volume of a Euglena cell body dispersion to be subjected to the anaerobic culture step to the volume of the culture product can be 1/10 or less, preferably 1/20 or less, particularly preferably 1/50 or less, and further preferably 1/75 or less, the content of the organic acid after completion of the anaerobic culture step preferably further increases. It is to be noted that the concentration method is not limited to centrifugation, and concentration may also be carried out by other known methods, such as a method of accumulating a dispersoid by filtration using a filter.

As a medium used in the anaerobic culture step, the same medium as that used in the above-described nitrogen-deficient culture step and heterotrophic culture step may be used. Otherwise, a buffer that does not contain a nutrient source for promoting the growth of Euglena may also be used. Examples of such a buffer that can be used in the culture include a HEPES buffer, a Tris buffer, a PBS buffer, a MOPS buffer, and a MES buffer. Moreover, as culture conditions for the anaerobic culture step, the same culture conditions as those for the above-described nitrogen-deficient culture step and heterotrophic culture step may be applied, with the exception that air bubbling is not carried out. Shaking culture is preferably carried out because the content of an organic acid in the culture medium further increases after completion of the anaerobic culture step.

The anaerobic culture is carried out preferably fur at least 1 day or more, more preferably for 2 days or more, and particularly preferably for 3 days or more. By subjecting Euglena to the anaerobic culture the Euglena releases an organic acid such as succinic acid into the culture medium. After completion of the anaerobic culture, the culture medium comprises succinic acid, which is in a non-concentrated state immediately after completion of the culture, in a concentration of 100 mg/L or more, preferably 200 mg/L or more, and particularly preferably 300 mg/L or more. The content of succinic acid in the culture medium after completion of the anaerobic culture can also be increased to 800 mg/L, or more, or 1000 mg/L or more, or further to 1500 mg/L or more, by optimization of the culture conditions. Therefore, after completion of the anaerobic culture, the culture medium can comprise an organic acid, which is in a non-concentrated state immediately after completion of the culture, in a concentration of 100 mg/L or more, 200 mg/L or more, 300 mg/L or more, 800 mg/L or more, 1000 mg/L or more, or 1500 mg/L or more. Since the produced organic acid (which is, in particular, succinic acid) has antibacterial action, it can prevent the growth of specific bacteria, etc.

The content of an organic acid in the culture medium after completion of the anaerobic culture can be obtained by analyzing, for example, with HPLC, a sample obtained by drying a supernatant that has been collected by centrifugation of the culture product. The weight percentage of the organic acid contained in the supernatant to the dry weight of the Euglena cell body after completion of the anaerobic culture is preferably 0.5% by weight or more, particularly preferably 1% by weight or more, and further preferably 1.5% by weight or more.

Examples of the organic acid contained in the culture medium after completion of the anaerobic culture include citric acid, malic acid, and lactic acid, as well as succinic acid. In many cases, the content of such organic acids other than citric acid is extremely small in comparison to the content of succinic acid. The amounts of malic acid and lactic acid contained in the culture medium after completion of the anaerobic culture may each be ⅕ or less, preferably ⅙ or less, particularly preferably 1/7, and further preferably 1/10 or less of the content of succinic acid. From the viewpoint of separation and purification of succinic acid having high usefulness, the amounts of such malic acid and lactic acid are preferably small. Moreover, the culture medium after completion of the anaerobic culture can comprise the following other useful compounds: 3-hydroxypropionic acid, malonic acid, propionic acid, serine, acetoin, aspartic acid, fumaric acid, 3-hydroxybutyrolactone, threonine, arabinitol, furfural, glutamic acid, itaconic acid, levulinic acid, proline, xylitol, xylonic acid, aconitic acid, citric acid, 2,5-furandicarboxylic acid, glucaric acid, lysine, levoglucosan, and sorbitol. The content of such compounds may be ⅕ or less, preferably ⅙ or less, particularly preferably 1/7, and further preferably 1/10 or less of the content of succinic acid.

With regard to the culture product obtained after completion of the anaerobic culture, an organic acid is contained not only in the culture medium, but also in the Euglena cell body. The organic acid contained in the Euglena cell body can be collected by extracting it with water or an organic solvent. Upon the extraction, the cell membrane may be destructed by giving mechanical force using an ultrasonic cell disintegrator or the like, or by adding enzyme such as lysozyme. It is to be noted that the above-described useful compounds, as well as an organic acid, may be comprised also in the Euglena cell body.

High-purity organic acid can be purified from the culture product containing the organic acid, which has been obtained after completion of the anaerobic culture, according to a conventionally known method. For instance, from a product obtained by drying a supernatant collected by centrifugation of the culture product, high-purity succinic acid can be purified by desalinating it with an ion exchange resin and then performing means such as crystallization or column chromatography.

With regard to the Euglena cell body in the culture product obtained after completion of the anaerobic culture, not only an organic acid contained therein, but also the cell body itself has usefulness. The Euglena cell body has been known to contain a large number of vitamin, mineral, amino acid, unsaturated fatty acid, etc., and thus, the Euglena cell body is useful as a food product, a dietary supplement, or a feed. In addition, in the case of the Euglena cell body obtained after completion of the anaerobic culture by the method of the present invention, the amount of carbohydrate contained in the cell body is reduced during the anaerobic culture. Hence, according to the method of the present invention, it becomes possible to provide a Euglena cell body containing a lower amount of carbohydrate, which has a higher value than a conventional one. Therefore, in another aspect, the present invention also relates to a Euglena cell body obtained by a method, which comprises the above-described nitrogen-deficient culture step or heterotrophic culture step, and the above-described anaerobic culture step, and further comprises a step of collecting the Euglena cell body from the culture product obtained by the anaerobic culture step according to centrifugation or the like.

Moreover, as described above, in the method of the present invention, the culture medium after completion of the anaerobic culture step comprises an organic acid, which is in a non-concentrated state immediately alter completion of the culture, in a concentration of 100 mg/L or more, preferably 200 mg/L or more, and particularly preferably 300 mg/L or more. More preferably, the culture medium after completion of the anaerobic culture step comprises succinic acid, which is in a non-concentrated state immediately after completion of the culture, in a concentration of 100 mg/L or more, preferably 200 mg/L or more, and particularly preferably 300 mg/L or more. Therefore, in a further aspect, the present invention relates to a culture product, and in particular, a culture medium, which is obtained by culturing Euglena comprising an organic acid (succinic acid) produced thereby in a concentration of 100 mg/L, or more. A culture medium obtained after culturing other algae including Euglena, which comprises such a large amount of organic acid, has not yet been known so far.

### EXAMPLES

Hereinafter, the present invention will be described in more detail in the following examples. However, these examples are not intended to limit the scope of the present invention.

### 1. Test Procedures

### (1) Culture of Euglena (Under Nitrogen-Sufficient or -Deficient Conditions)

Wild-type Euglena gracilis (which had been obtained from EUGLENA CO, LTD,, and had been then pre-cultured in an ordinary culture environment, such as under nitrogen-sufficient conditions, for approximately 1 to 2 weeks) was subjected to an aerobic and static culture in an incubator, by setting the initial Euglena cell body concentration at 10 to 20 g/L, using a modified Cramer-Myers medium (modified CM medium, pH 3.5) having the composition shown in Table 1, at 25° C. for 12 hours in a light-dark cycle, while adding air containing 1% CO₂ into the incubator by air bubbling. The light intensity was set at 50 to 100 μmol m⁻²s⁻¹. The culture was carried out using a 1-L culture vessel. When the culture was carried out under nitrogen-deficient conditions, (NH₄)₂HPO₄ used as a nitrogen source was removed from the composition of the modified CM medium.

TABLE 1

| Reagent | Amount per L |
|---|---|
| (NH₄)₂HPO₄ | 1 g |
| KH₂PO₄ | 1 g |
| Na₃C₆H₅O₇•2H₂O | 0.52 mg |
| Fe₂(SO₄)₃•6H₂O | 3 mg |
| MnCl₂•4H₂O | 1.8 mg |
| Co(NO₃)₂•6H₂O | 1.3 mg |
| ZnSO₄•7H₂O | 0.4 mg |
| H₂MoO₄ | 0.2 mg |
| CuSO₄•5H₂O | 0.02 mg |
| Thiamin hydrochloride | 0.01 mg |
| Vitamin B₁₂ | 0.05 mg |
| MgSO₄•7H₂O | 12 mg |
| CaCl₂•2H₂O | 26.6 mg |

### (2) Anaerobic Culture

A predetermined amount of the culture medium obtained in the above (1) was collected, and was then centrifuged (5000 rpm×5 minutes). Thereafter, a supernatant was discarded. The obtained Euglena cell body was suspended in 10 mL of 20 mM HEPES-KOH buffer (pH 7.8), and the suspension was then transferred into a 20-mL volume vial bottle. The vial bottle was capped with a rubber stopper, and two injection needles were then inserted into the stopper. From the one needle, nitrogen gas was introduced into the bottle for 1 hour. During the introduction of nitrogen gas, the vial bottle was left in a light irradiation environment at 30° C. One hour later, the injection needles were removed, and the vial bottle, which was light-shielded with aluminum foil, was placed in an incubator at 25° C., and was then subjected to a shaking culture. Three days or six days after the initiation of the shaking culture, the bottle was uncapped, and the content was transferred into a 15-mL tube, and was immediately subjected to centrifugation (10,000 rpm×2 minutes). The obtained supernatant was transferred into another tube, and was then freeze-dried.

The dry weight of the Euglena cell body before being subjected to an anaerobic culture was calculated, separately, by collecting 50 mL of the culture medium obtained in the above (1) and placing it into a tube, then discarding a supernatant after centrifugation, then washing the cell body with sterile water twice to remove salts, then freeze-drying the resultant at −80° C. for 1 day or more, and then measuring the weight of the resulting product.

### (3) Quantification of an Organic Acid

An analysis was carried out according to a post-labelling method using bromothymol blue. A freeze-dried product of the supernatant obtained in the above (2) was dissolved in 100 μL of 3 mM filtrated perchloric acid aqueous solution, and was then analyzed using high performance liquid chromatograph (LC-2000Plus, manufactured by JASCO CORPORATION) equipped with a photodiode array detector and two columns (RSpak KC-811, manufactured by SHOW DENKO K. K.). Using 0.2 mM bromothymol blue solution (in 15 mM sodium phosphate buffer), an organic acid was quantified, and then, a peak was detected at a wavelength of 445 nm. The column temperature was set at 60° C., and the flow rates of the 3 mM perchloric acid aqueous solution and the 0.2 mM bromothymol blue solution were set at 1.0 mL/min and 1.5 mL/min, respectively.

### 2. Test Results

### (1) Test 1

800 mL of a culture medium, which had been obtained by being cultured under nitrogen-sufficient conditions, or under nitroven-deficient conditions for 11 days, was centrifuged, and the obtained Euglena cell body was then suspended in 10 mL of a buffer, followed by performing an anaerobic culture for 3 days. After completion of the anaerobic culture, the amounts of organic acids contained in the culture medium are shown in Table 2. Even though the Euglena cultured under nitrogen-sufficient conditions was subjected to an anaerobic culture, almost no succinic acid was contained in the obtained supernatant, the amount of the succinic acid to the dry weight of the cell body before the anaerobic culture was only 0.08% by weight. On the other hand, when Euglena was cultured under nitrogen-deficient conditions for 11 days and was then subjected to an anaerobic culture, a large amount of succinic acid was contained in the obtained supernatant, and the amount of the succinic acid to the dry weight of the cell body before the anaerobic culture was 1.9% by weight. This amount corresponded to an amount that was 20 times or more greater than the amount of succinic acid contained in a sample cultured under nitrogen sufficient conditions (relative to the dry weight of the cell body before the anaerobic culture). It is to be noted that citric acid could not be quantified because its peak in the HPLC chart was overlapped with those of other components. However, from the chart, it was assumed that citric acid would have been produced in an amount equivalent to that of succinic acid.

TABLE 2

| | | Citric | | | |
|---|---|---|---|---|---|
| | Succinic acid | acid | Malic acid | Lactic acid | Dry weight |
| Sample | (mg) | (mg) | (mg) | (mg) | (g) |
| Nitrogen- | 12 ± 2 | N/I | ND | ND | 14.8 ± 1.4 |
| sufficient | | | | | |
| Nitrogen- | 870 ± 270 | N/I | 140 ± 70 | ND | 45.5 ± 10.0 |
| deficient | | | | | |
| (11 days) | | | | | |

Organic acid: weight per L of culture medium after anaerobic culture;
dry weight: weight of cell body per L of culture medium before anaerobic culture;
N/I: not identified due to overlapping with peaks of other components;
number of samples: n = 3

### (2) Test 2

800 mL of a culture medium, which had been obtained by being cultured under nitrogen-deficient conditions for 11 days or for 14 days, was centrifuged, and the obtained Euglena cell body was then suspended in 10 mL of a buffer, followed by performing an anaerobic culture for 3 days. After completion of the anaerobic culture, the amounts of organic acids contained in the culture medium are shown in Table 3, in both types of the number of culture days, a large amount of succinic acid was contained in the obtained supernatant, and the amounts of the succinic acid in both cases to the dry weight of the cell body before the anaerobic culture were 75% by weight and 3.1% by weight, respectively.

TABLE 3

| | | Citric | | | |
|---|---|---|---|---|---|
| | Succinic acid | acid | Malic acid | Lactic acid | Dry weight |
| Sample | (mg) | (mg) | (mg) | (mg) | (g) |
| Nitrogen- | 1760 | N/I | 160 | 20 | 23.3 |
| deficient | | | | | |
| (11 days) | | | | | |
| Nitrogen- | 1340 | N/I | 110 | 40 | 42.7 |
| deficient | | | | | |
| (14 days) | | | | | |

Organic acid: weight per L of culture medium after anaerobic culture;
dry weight: weight of cell body per L of culture medium before anaerobic culture;
N/I: not identified due to overlapping with peaks of other components;
number of samples: n = 1

### (3) Test 3

800 mL of a culture medium, which had been obtained by being cultured under nitrogen-deficient conditions for 14 days, was centrifuged, and the obtained Euglena cell body was then suspended in 10 mL of a buffer, followed by performing an anaerobic culture for 6 days. After completion of the anaerobic culture, the amounts of organic acids contained in the culture medium are shown in Table 4. Comparing the obtained results with the results of Test 2, even if the number of days fir the anaerobic culture was increased from 3 days to 6 days, a great difference was not found in the content of succinic acid. The content of the succinic acid to the dry weight of the cell body before the anaerobic culture was 4.8% by weight. It was assumed that a deviation in the contents of succinic acids in the results of Tests 1 to 3 would be caused by differences in the position of the culture vessel in the incubator, the degree of light irradiation associated with the position of the culture vessel, and the like.

TABLE 4

| | Succinic | | | Lactic | Dry |
|---|---|---|---|---|---|
| | acid | Citric acid | Malic acid | acid | weight |
| Sample | (mg) | (mg) | (mg) | (mg) | (g) |
| Nitrogen- | 1590 ± | 1240 ± 0 | 150 ± 20 | 110 ± 20 | 32.9 ± 6.0 |
| deficient | 110 | | | | |
| (14 days) | | | | | |
| (Anaerobic | | | | | |
| culture | | | | | |
| (6 days)) | | | | | |

Organic acid: weight per L of culture medium after anaerobic culture;
dry weight: weight of cell body per L of culture medium before anaerobic culture;
number of samples: n = 3

### (4) Test 4

20 to 100 mL of a culture medium, which had been obtained by being cultured under nitrogen-sufficient conditions, using a CM medium containing 10 mM sodium acetate, or using a control CM medium containing no additives, for 14 days, was centrifuged. The obtained Euglena cell body was suspended in 10 mL of a buffer, followed by performing an anaerobic culture for 3 days. It is to be noted that the culture under nitrogen-sufficient conditions was carried out, while the light intensity was set at 200 mol μmol m⁻²s⁻¹. In the case of the control, the content of succinic acid to the dry weight of the cell body before the anaerobic culture was 1.1% by weight, whereas the content of the succinic acid was 1.5% by weight in the case of adding sodium acetate to the medium. It is considered that the amount of succinic acid produced was smaller than those in other test examples, since the amount of the used Euglena cell body was small.

TABLE 5
Nitrogen-sufficient (14 days), anaerobic culture (3 days)

| | Succinic acid | Dry weight |
|---|---|---|
| Sample | (mg) | (g) |
| Control | 29.9 | 2.7 |
| +10 mM sodium acetate | 42.7 | 2.8 |

Succinic acid: weight per L of culture medium after anaerobic culture
Dry weight: weight of cell body per L of culture medium before anaerobic culture,
number of samples: n = 2

All publications, patents and patent applications cited in the present description are incorporated herein by reference in their entirety.

1. A method for producing an organic acid using Euglena, which comprises a nitrogen-deficient culture step of aerobically culturing Euglena under nitrogen-deficient conditions, and an anaerobic culture step of incubating a culture product obtained in the nitrogen-deficient culture step under anaerobic conditions.

2. The method according to claim 1, wherein the nitrogen-deficient culture step is carried out for 3 days or more.

3. A method for producing an organic acid using Euglena, which comprises a heterotrophic culture step of aerobically culturing Euglena using a medium containing a carbon source, and an anaerobic culture step of incubating a culture product obtained in the heterotrophic culture step under anaerobic conditions.

4. The method according to claim 1, which further comprises a step of collecting a culture medium containing the organic acid from the culture product obtained in the anaerobic culture step.

5. The method according to claim 1, which further comprises a step of concentrating a Euglena cell body contained in the culture product obtained in the nitrogen-deficient culture step or the heterotrophic culture step, before performing the anaerobic culture step.

6. The method according to claim 1, wherein the Euglena is wild-type Euglena.

7. The method according to claim 1, wherein the organic acid is succinic acid.

8. The method according to claim 3, which further comprises a step of collecting a culture medium containing the organic acid from the culture product obtained in the anaerobic culture step.

9. The method according to claim 3, which further comprises a step of concentrating a Euglena cell body contained in the culture product obtained in the nitrogen-deficient culture step or the heterotrophic culture step, before performing the anaerobic culture step.

10. The method according to claim 3, wherein the Euglena is wild-type Euglena.

11. The method according to claim 3, wherein the organic acid is succinic acid.
